(19) United States
(12) Patent Application Publication
(10) Pub. No.: US 2009/0194114 A1
(43) Pub. Date: Aug. 6, 2009

(54) Laryngeal Mask Airway Installation Kit, Clip Device, and Laryngeal Mask Airway

(76) Inventors: Tien-Sheng Chen, Taipei City (TW); Hui-Bih Yuan, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/342,224

(22) Filed: Dec. 23, 2008

(30) Foreign Application Priority Data
Jan. 31, 2008 (TW) 097103784

(51) Int. Cl. A61M 16/06

(52) U.S. Cl. 128/207.15; 128/202.13

(57) ABSTRACT

A laryngeal mask airway installation kit of the invention comprises a laryngeal mask airway and a clip device. The laryngeal mask airway comprises a gas filled portion, an against portion, and a tube portion. The gas filled portion connects to one end of the tube portion, and the against portion is connected to the tube portion. The clip device comprises a main portion and a handle portion. At least part of the main portion is connected to the against portion, and the main portion presses the gas filled portion of the laryngeal mask airway. The handle portion is connected to the main portion. When the main portion presses the gas filled portion of the laryngeal mask airway, the handle portion is neighbor to the tube portion.

### BACKGROUND OF THE INVENTION

1. Field of the Invention

The present invention relates to a medical instrument; more particularly, the present invention relates to a laryngeal mask airway installation kit, clip device, and laryngeal mask airway.

2. Description of the Related Art

In the case of anaesthesia or apnea patients, the top priority is the provision of pulmonary ventilation by emergency medical personnel. Among the various respiration sustaining instruments, the laryngeal mask airway (LMA), due to its ease of operability, is a common alternative to endotracheal intubation.

Please refer to FIG. 1 for the structure of a conventional LMA 10P; basically, the LMA 10P mainly comprises a flexible tubular portion 11P, an inflatable sealing cuff 13P, and an inflation tube 15P, wherein the flexible tubular portion 11P comprises a first opening part 111P and a second opening part 112P, the inflatable sealing cuff 13P is disposed surrounding the first opening part 111P, and the inflation tube 15P has one end connected to the inflatable sealing cuff 15P and the other end directing gas into the inflatable sealing cuff 13P.

Please refer to FIGS. 2 to 4 for illustrative diagrams showing the conventional laryngeal mask 10P in use. First, a user may insert the LMA 10P into a patient's mouth by the end having the inflatable sealing cuff 13P, which is in a deflated condition. When the inflatable sealing cuff 13P reaches a deeper part of the mouth, the user will then have to insert his/her finger into the patient's mouth to bend the front end of the LMA 10P to make the structure of the LMA 10P conform with the structure of the patient's upper jaw. In addition, due to the softness of the LMA 10P, the user may not exert force thereby; thus, the user has to push aside the tongue with his/her finger to bend the first opening part 111P and the flexible tubular portion 11P to such an extent that they can reach past the upper jaw to the opening of the trachea, as shown in FIG. 3. In FIG. 4, it is shown that when the LMA 10P gets to a specific position, the user may then aerate the inflatable sealing cuff 13P from the inflation tube 15P so as to form a sealing mask in the patient's throat. The sealing mask may encompass the opening of the trachea and form an air passage thereby; after that, the user may direct gas, such as oxygen, from the second opening part 112P to maintain the patient's respiration.

Accordingly, during the installation of an LMA, users always have to insert their finger(s) (especially their index fingers) into a patient's mouth to pass the soft, inconvenient LMA through the upper jaw because of the obstruction caused by the patient's tongue. In general, this displacement of fingers may cause two problems. First, there is the risk that the user may be bitten by the patient during the installation of an LMA. Second, in a case where a patient's oral space is overly small, which may be caused by an overly small mouth, an overly tight jaw joint, or an overly thick tongue, a user may encounter difficulty inserting his/her finger(s) into the patient's mouth, resulting in failure to install the LMA.

Therefore, it is desirable to provide a laryngeal mask airway installation kit, clip device, and laryngeal mask airway to mitigate and/or obviate the aforementioned problems.

### SUMMARY OF THE INVENTION

It is an object of the present invention to provide a laryngeal mask airway installation kit which could be easily installed into a patient's body.

It is another object of the present invention to provide a clip device for facilitating a laryngeal mask airway to be installed into a patient's body.

It is still another object of the present invention to provide a laryngeal mask airway having an against portion.

To achieve the aforementioned objects, a laryngeal mask airway (LMA) installation kit of the present invention comprises an LMA and a clip device. The LMA comprises a gas filled portion, an against portion, and a tube portion. The gas filled portion is connected to one end of the tube portion, and the against portion is connected to the tube portion. The clip device comprises a main portion and a handle portion. At least part of the main portion is connected to the against portion, and the main portion presses the gas filled portion of the LMA. The handle portion is connected to the main portion. When the main portion presses the gas filled portion of the LMA, the handle portion is neighbor to the tube portion.

According to one of the preferred embodiments of the present invention, the main portion is in the form of a curved surface, and its shape is egg-shaped. The main portion is connected to the against portion by means of a hook-shaped connection unit.

Other objects, advantages, and novel features of the invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIGS. 1 to 4 are illustrations of a laryngeal mask airway (LMA) used in the prior art.

FIG. 5 is an illustration of a first embodiment of an LMA installation kit of the present invention.

FIG. 6 is an exploded view of the first embodiment of the LMA installation kit of the present invention.

FIG. 7 is an illustration of a first embodiment of an LMA of the present invention.

FIG. 8 is an illustration of another embodiment of the LMA of the present invention.

FIG. 9 is an illustration of a second embodiment of the LMA installation kit of the present invention.

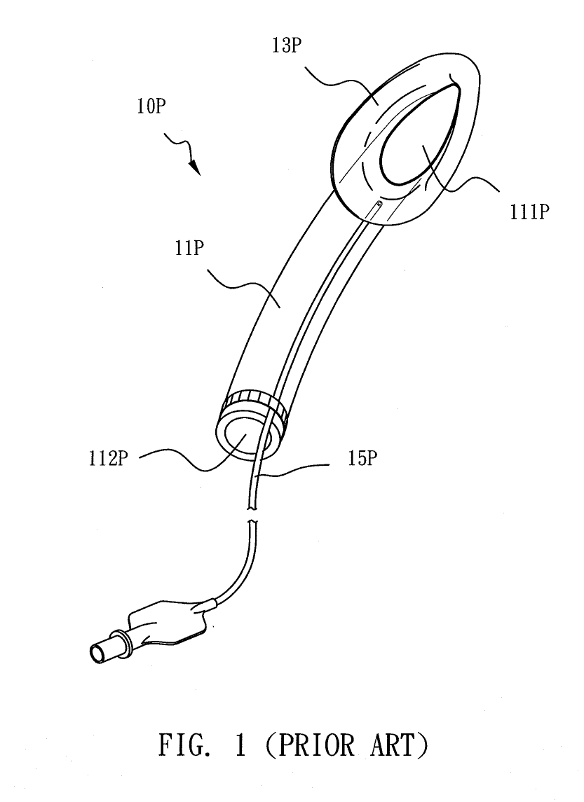

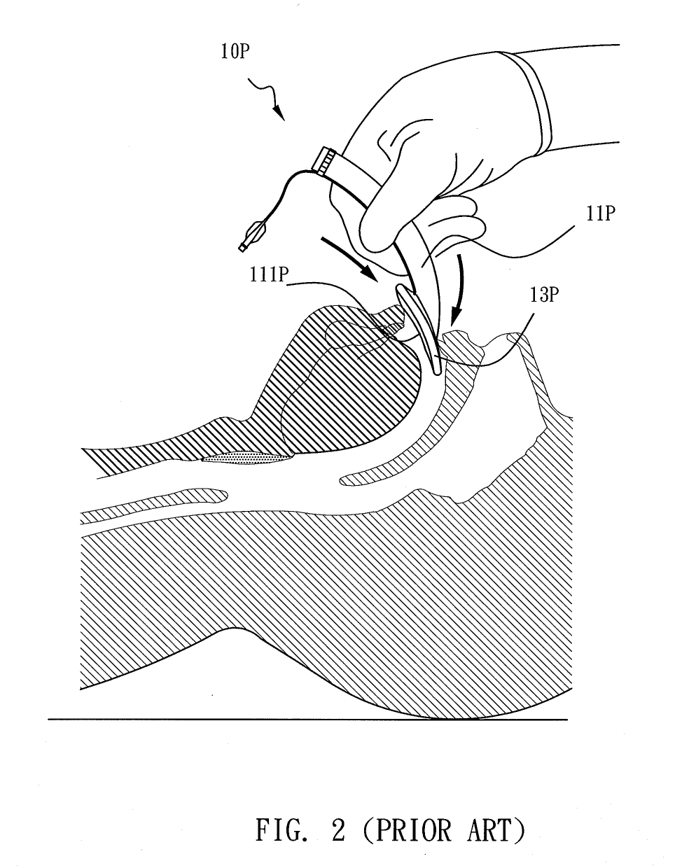

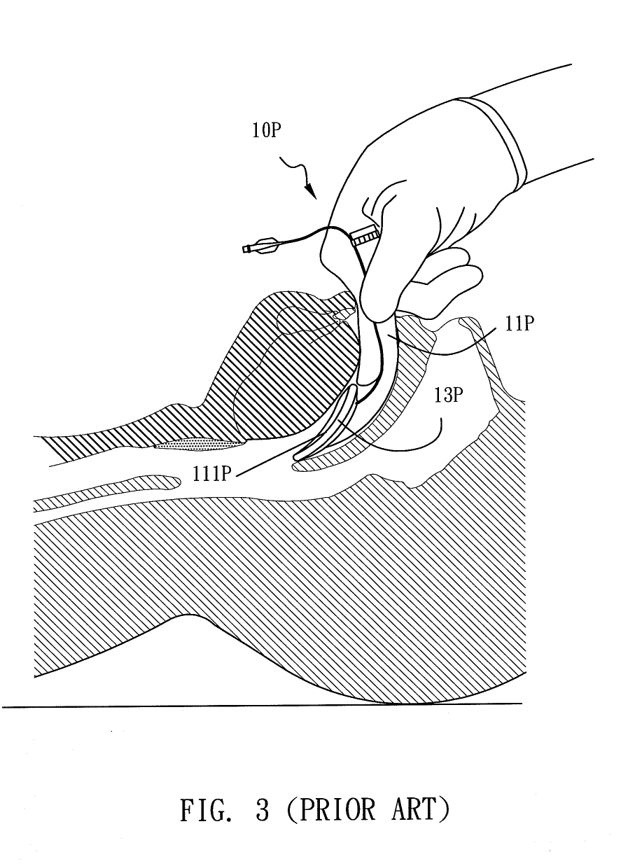

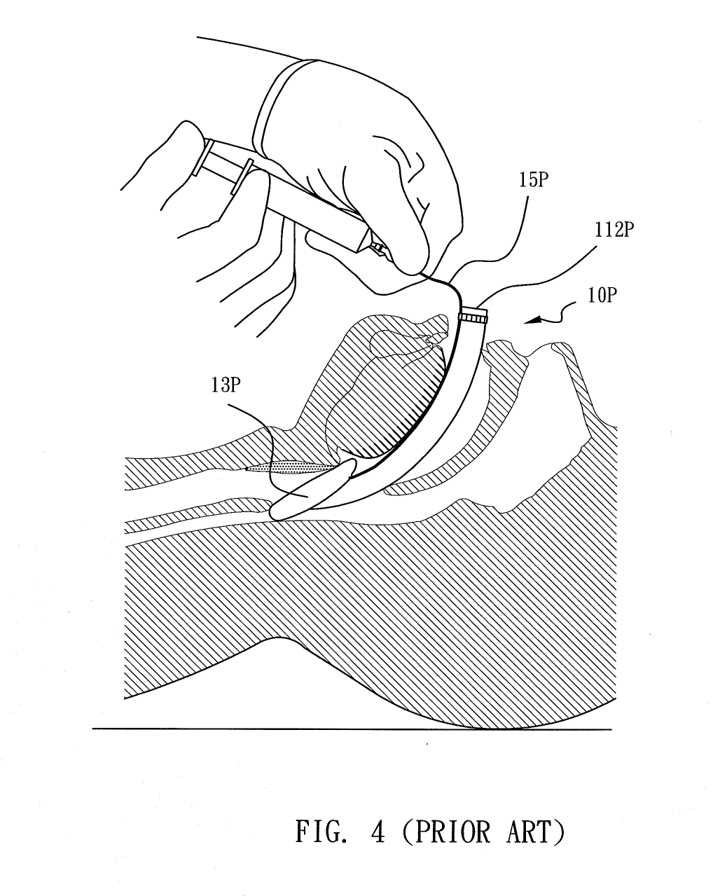

### DETAILED DESCRIPTION OF THE PREFERRED EMBODIMENT

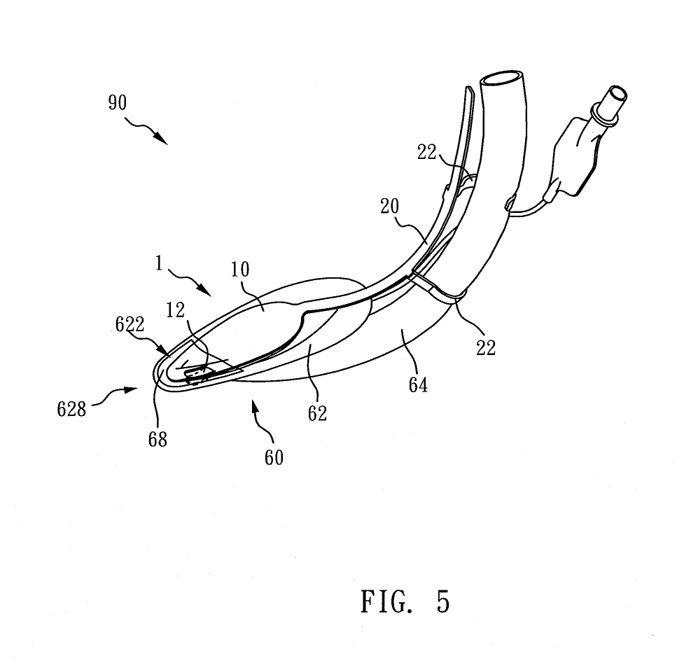

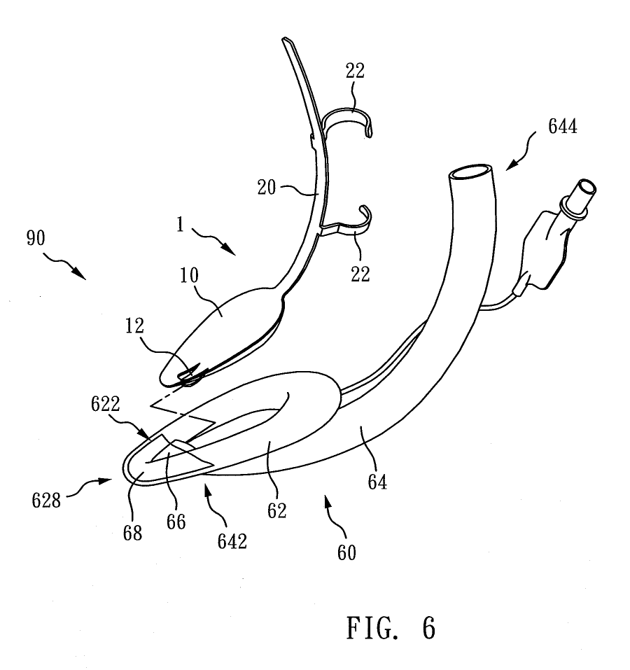

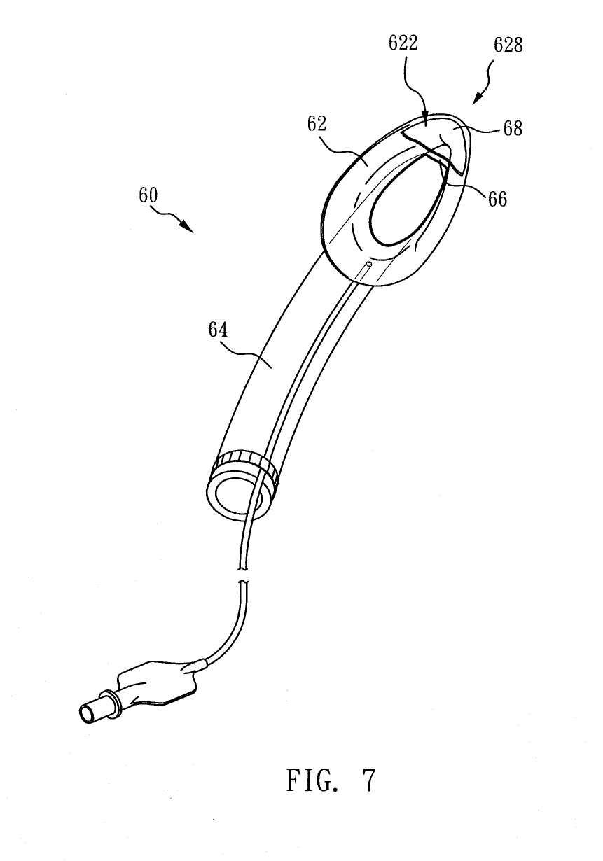

Please refer to FIGS. 5˜7. FIG. 5 is an illustration of a first embodiment of a laryngeal mask airway (LMA) installation kit of the present invention. FIG. 6 is an exploded view of the first embodiment of the LMA installation kit of the present invention. FIG. 7 is an illustration of a first embodiment of an LMA of the present invention.

The LMA installation kit 90 of the present invention comprises an LMA 60 and a clip device 1 associated with the LMA 60. The clip device 1 can connect to the LMA 60 (as shown in FIG. 5), and the clip device 1 can easily separate from the LMA 60 (as shown in FIG. 6).

The LMA 60 comprises a gas filled portion 62, a tube portion 64, an against portion 66 and a protection cover 68. The tube portion 62 comprises a first end 642 and a second end 644. The gas filled portion 62 is connected to the first end 642 of the tube portion 64 (as shown in FIG. 6). The gas filled portion 62 is in the form of a circular hollow structure capable of storing gas therein. When the gas filled portion 62 is inflated with a predetermined volume of gas, the exterior of the gas filled portion 62 forms a soft surface. The second end 644 of the tube portion 64 can be connected to an oxygen supply apparatus for the direct transmission of oxygen into a patient's trachea. Because the gas filled portion 62, when filled with gas, presses the periphery of the opening of the patient's trachea, the oxygen transmitted to the trachea will not leak from the opening of the patient's trachea.

The against portion 66 is located at a tail portion 628 of the gas filled portion 62. The purpose of setting the against portion 66 is to connect to the clip device 1. The gas filled portion 62 comprises an upper side 622 and a lower side 624, wherein the upper side 622 is the part capable of directly contacting the patient. In this embodiment, the against portion 66 is connected to the tube portion 64. More precisely, the against portion 66 is located at the intersection of the gas filled portion 62 and the tube portion 64 (as shown in FIG. 7).

The protection cover 68 is a soft material, and is connected to the tail portion 628 of the gas filled portion 62. In this embodiment, the protection cover 68 covers part of the upper side 622. Part of a main portion 10 could be placed in the protection cover 68. There are two purposes of setting the protection cover 68: One is to prevent the main portion 10 from directly contacting the trachea and thus injuring the patient's tissue; the other is to improve the operation when a user uses the clip device 1. In this embodiment, the protection cover 68 is connected to the edge of the tail portion 628 of the gas filled portion 62, and its surface area is greater than that of the against portion 66.

The clip device 1 comprises a main portion 10 and a handle portion 20. When the clip device 1 is connected to the LMA 60, the clip device 1 can strengthen the structure of the LMA 60, such that the LMA 60 can smoothly slide into the patient's body, and the patient's tongue can be lifted slightly by utilizing the main portion 10 of the clip device 1.

The main portion 10 comprises a connection unit 12. By using the connection unit 12 to connect to the against portion 66, the main portion 10 presses the gas filled portion 62 of the LMA 60. In this embodiment, the main portion 10 can press the upper side 622 of the gas filled portion 62.

The purpose of setting the connection unit 12 is to connect to the against portion 66. Therefore, the shape of the connection unit 12 has to match that of the against portion 66. In this embodiment, the connection unit 12 is in the form of a hook. When the connection unit 12 connects to the against portion 66, the periphery of the main portion 10 can press the upper side 622 of the gas filled portion 62 (as shown in FIG. 5).

Please note that the main portion 10 is not always required to have the connection unit 12. For example, when the shape of the end of the main portion 10 matches that of the against portion 66, the main portion 10 does not need the connection unit 12, and can directly utilize the end of the main portion 10 to connect to the against portion 66.

The main portion of the clip device of the present invention could be in any shape. For example, the main portion could be in the form of a plane, a curved surface or a frame (such as an O-shaped frame or a U-shaped frame). Further, the shape of the main portion could be egg-shaped or oval-shaped so as to match that of the gas filled portion 62. In this embodiment, the main portion is in the form of a concave curved surface, and is egg-shaped (similar to the part of a spoon used for spooning liquids).

The handle portion 20 is connected to the main portion 10. When the main portion 10 presses the gas filled portion 62 of the LMA 60, the handle portion 20 is neighbor to the tube portion 64. More precisely, when the user (such as a doctor) operates the LMA installation kit 90, he/she can hold both the handle portion 20 and the tube portion 64 at the same time. In this embodiment, the handle portion 20 has a flexible characteristic, such that it can associate with the tube portion 64, which is also flexible.

In this embodiment, in order to connect the handle portion 20 to the tube portion 64, the handle portion 20 comprises at least one mounting element 22. The mounting element 22 is used for connecting to the tube portion 64. The shape of each mounting element 22 matches that of the tube portion 64. In this embodiment, the at least one mounting element 22 comprises two C-shaped clip elements, wherein the internal diameter of each C-shaped clip element is equal to or slightly bigger than the external diameter of the tube portion 64. Further, there is a predetermined distance between the two C-shaped clip elements, and their C-shaped openings are facing opposite directions (as shown in FIG. 6). By means of the action of these two C-shaped clip elements, the handle portion 20 can easily connect to the tube portion 64, and can easily separate from the tube portion 64 as well.

Please note that the handle portion 20 is not always required to have the mounting element 22. The user could directly hold both the handle portion 20 and the tube portion 64 so as to prevent them from being separated.

Please refer to the following description regarding the operating instructions of the LMA installation kit 90 of the present invention. Please refer to FIG. 6. First, the connection unit 12 of the main portion 10 of the clip device 1 is connected to the gas filled portion 62 and the against portion 66 of the LMA 60; then, the mounting element 22 of the handle portion 20 of the clip device 1 is connected to the tube portion 64 of the LMA 60, thereby becoming the operational status as shown in FIG. 5.

Next, the LMA installation kit 90 slides into the patient's throat through the tail portion 628. When the tail portion 628 enters past the patient's tongue, since the patient is usually lying down at this time, and the patient's tongue would droop due to gravity thereby obstructing the LMA 60 from entering the passage of the patient's trachea, the present invention would utilize the main portion 10 of the clip device 1 to slightly lift (against gravity) the patient's tongue, thus facilitating the passage of the LMA 60 into the patient's trachea.

At this time, the clip device 1 has accomplished the function of pushing aside the patient's tongue. Therefore, the clip device 1 could be removed from the LMA installation kit 90, while the LMA 60 continues sliding into the patient's larynx till the gas filled portion 62 presses the opening of the trachea. When the gas filled portion 62 presses the opening of the trachea, the circular gas filled portion 62 can completely cover the periphery of the opening of the trachea so as to form an independent respiratory passage. At this time, the oxygen supply apparatus can be connected to the tube portion 64 of the LMA 60 for transmitting oxygen to the patient's trachea via the tube portion 64.

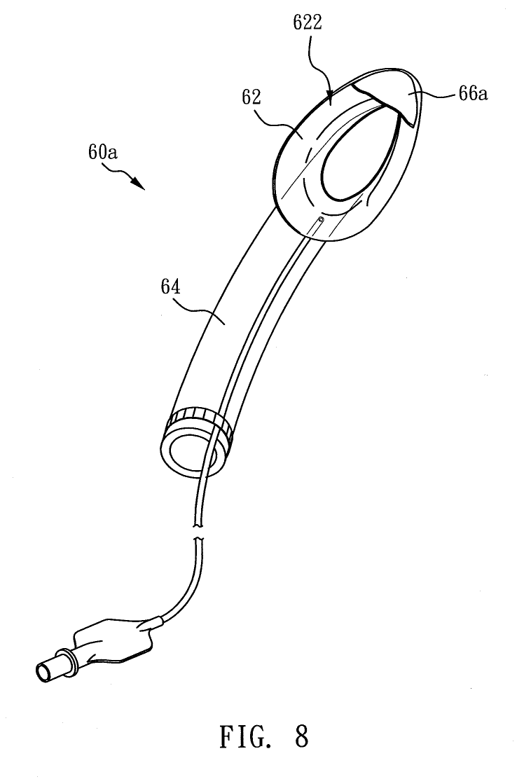

Please refer to FIG. 8 for an illustration of another embodiment of the LMA of the preset invention. The LMA 60a comprises a gas filled portion 62, a tube portion 64 and an against portion 66a. The main portion of the clip device (not shown in figures) associated with the LMA 60a can press the upper side 622 of the gas filled portion 62. The difference between the first and second embodiments is the position and size of the against portion 66a. In this embodiment, the against portion 66a is located at the upper side 622 of the gas filled portion 62, and the against portion 66a has a larger pressing area. Therefore, the shape and structure of the clip device associated with the LMA 60a are similar to those of the clip device 1 of the first embodiment (as shown in FIG. 6), while in this embodiment, the connection unit of the clip device is larger, and the position of the connection unit is closer to the end of the main portion of the clip device.

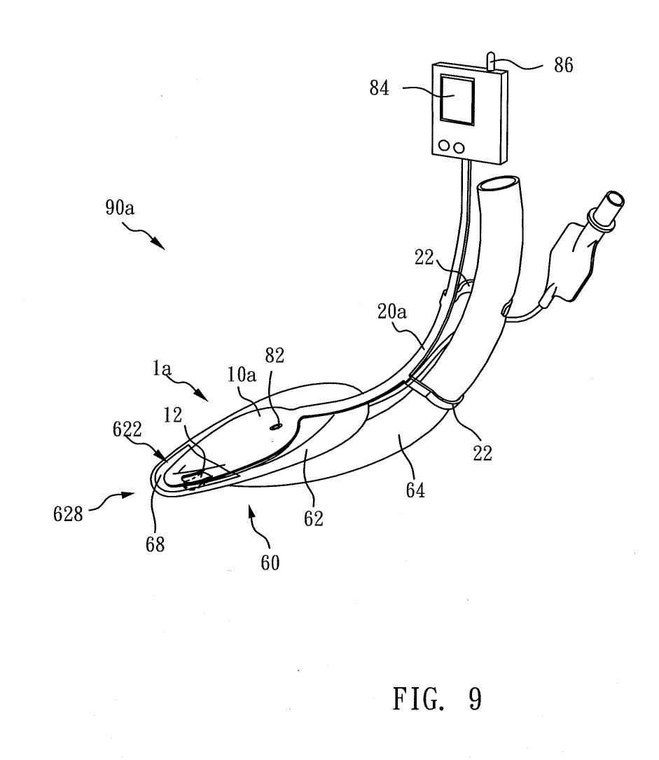

Please refer to FIG. 9 for an illustration of a second embodiment of the LMA installation kit of the present invention. The LMA installation kit 90a comprises an LMA 60 and a clip device 1a. The major difference between the first and second embodiments is that the clip device 1a comprises an image-capturing unit 82, a display unit 84 and a signal emission unit 86. The image-capturing unit 82 is used for capturing an image from a patient's upper airway. The captured image may then be directly shown on the display unit 84 connected to the handle portion 20a so that the user can determine the position of the LMA in the patient's upper airway. In addition, the image captured by the image-capturing unit 82, depending on the user's need, could be transmitted to an external display (not shown in figures) via the signal emission unit 86. Therefore, the LMA installation kit 90a of the present invention not only provides a function of facilitating the installation of the LMA 60 but also allows the user to conveniently observe the installation status, so as to be provided with a more accurate status evaluation. Please note that the position of the image-capturing unit 82 is not limited to the above description.

Although the present invention has been explained in relation to its preferred embodiments, it is to be understood that many other possible modifications and variations can be made without departing from the spirit and scope of the invention as hereinafter claimed.

What is claimed is:

1. A clip device for clipping a laryngeal mask airway, the laryngeal mask airway comprising a gas filled portion, an against portion and a tube portion, the clip device comprising:
a main portion, at least part of the main portion connecting to the against portion, the main portion pressing the gas filled portion of the laryngeal mask airway; and
a handle portion connected to the main portion; when the main portion presses the gas filled portion of the laryngeal mask airway, the handle portion is neighbor to the tube portion.

2. The clip device as claimed in claim 1, wherein the main portion is in the form of a plane, a curved surface or a frame.

3. The clip device as claimed in claim 2, wherein the shape of the main portion is egg-shaped or oval-shaped.

4. The clip device as claimed in claim 1, wherein the main portion further comprises a connection unit connected to the against portion.

5. The clip device as claimed in claim 4, wherein the connection unit is in the form of a hook.

6. The clip device as claim in claim 4, wherein the handle portion further comprises at least one mounting element connected to the tube portion.

7. The clip device as claimed in claim 6, wherein each mounting element is a C-shaped clip element.

8. The clip device as claimed in claim 1, wherein the clip device further comprises an image-capturing unit.

9. The clip device as claimed in claim 8, wherein the clip device further comprises a display unit displaying an image captured by the image-capturing unit.

10. The clip device as claimed in claim 8, wherein the clip device further comprises a signal emission unit transmitting an image captured by the image-capturing unit to an external display.

11. A laryngeal mask airway comprising:
a tube portion comprising a first end and a second end;
a gas filled portion connected to the first end of the tube portion, the gas filled portion comprising an upper side and a lower side; and
an against portion connected to the tube portion.

12. The laryngeal mask airway as claimed in claim 11, wherein the against portion is substantially located at the intersection of the gas filled portion and the tube portion.

13. The laryngeal mask airway as claimed in claim 11, wherein the laryngeal mask airway further comprises a protection cover connected to the gas filled portion.

14. The laryngeal mask airway as claimed in claim 13, wherein the protection cover covers part of the upper side of the gas filled portion.

15. A laryngeal mask airway installation kit comprising:
a laryngeal mask airway comprising:
a tube portion comprising a first end and a second end;
a gas filled portion connected to the first end of the tube portion, the gas filled portion comprising an upper side and a lower side;
a protection cover connected to the gas filled portion, wherein the protection cover covers part of the upper side of the gas filled portion; and
an against portion connected to the tube portion; and
a clip device comprising:
a main portion, at least part of the main portion connecting to the against portion, the main portion pressing the gas filled portion of the laryngeal mask airway; and
a handle portion, connected to the main portion; when the main portion presses the gas filled portion of the laryngeal mask airway, the handle portion is neighbor to the tube portion.

16. The laryngeal mask airway installation kit as claimed in claim 15, wherein the against portion is substantially located at the intersection of the gas filled portion and the tube portion.

17. The laryngeal mask airway installation kit as claimed in claim 15, wherein the main portion presses the upper side of the gas filled portion.

18. The laryngeal mask airway installation kit as claimed in claim 15, wherein the main portion is in the form of a plane, a curved surface or a frame.

19. The laryngeal mask airway installation kit as claimed in claim 15, wherein the main portion further comprises a connection unit connected to the against portion.

20. The laryngeal mask airway installation kit as claimed in claim 15, wherein the handle portion further comprises at least one mounting element connected to the tube portion.
